(19) United States
(12) Patent Application Publication
(10) Pub. No.: US 2004/0215061 A1
(43) Pub. Date: Oct. 28, 2004

(54) Visualization stylet for endotracheal intubation

(76) Inventors: Zebadiah Kimmel, Chicago, IL (US); Raymond Glassenberg, Chicago, IL (US)

Correspondence Address: BELL & ASSOCIATES, 416 FUNSTON ST., SUITE 100, SAN FRANCISCO, CA, 94118, US

(21) Appl. No.: 10/833,571

(22) Filed: Apr. 27, 2004

Related U.S. Application Data
(60) Provisional application No. 60/465,976, filed on Apr. 28, 2003.

(51) Int. Cl. A61B 1/06; A61B 1/267

(52) U.S. Cl. 600/179; 600/160; 600/120

(57) ABSTRACT

An endotracheal visualization stylet having a self-contained light source and camera, providing ease of use, economy of manufacture and superior optics.

This application claims the benefit of co-pending United States provisional application serial No. 60/465,976 filed Apr. 28^{th}, 2003.

### FIELD OF THE INVENTION

The invention relates to medical devices and methods used to visualize the interior anatomy of a body cavity. Specifically, the invention relates to devices used to illuminate and visualize the interior of the oral cavity and larynx during endotracheal intubation.

### BACKGROUND

In the course of providing medical care, particularly in an emergent situation or during anesthesia, it is frequently necessary to insert a tube into a patient's trachea to allow anesthesia and/or for the mechanical ventilation of the lungs of the patient. This procedure is called endotracheal intubation. It is important that the endotracheal tube be placed into the patient's trachea, rather than into the patient's esophagus (or anywhere else), otherwise air will not be delivered to the lungs. For this reason, it is important to be able to visualize the patient's glottis during endotracheal intubation. Improper endotracheal intubation is a significant cause of morbidity and mortality during anesthesia.

Typically, a device called a laryngoscope is used to facilitate endotracheal intubation. This device consists of a handle and a blade. There is the straight blade (“Miller blade”), and the slightly curved blade (“Macintosh blade”). The epiglottis normally overlies the glottic opening into the larynx to prevent the passage of food into the trachea during eating; therefore, in endotracheal intubation, it is necessary to displace the epiglottis from the glottic opening to permit the endotracheal tube to be inserted into the trachea. The blade is inserted into the patient's mouth and is used to lift the patient's tongue and epiglottis out of the way so that the patient's glottis (the entrance to the trachea) may be visualized, and the endotracheal tube may be inserted successfully into the trachea.

In some patients, such as obese patients or patients with atypical anatomy, the laryngoscope alone is unable to provide a clear view of the patient's glottis. So-called “blind intubation” may be attempted in such patients, but the failure rate of blind intubation is high. Blind intubation frequently leads to trauma and bleeding of the mucosa of the larynx and successful intubation may often require several attempts, slowing critical care and jeopardizing the patient's health. In such patients, therefore, it is desirable to place a visualization and/or an illumination device into the patient's pharynx to provide for a view of the glottis and allow proper insertion of the endotracheal tube.

In some cases, oral intubation is not desirable or practicable and a nasal intubation must be used. Three main techniques are used for nasal intubation. One method is to use an oral laryngoscope to observe and monitor the placement of the nasal endotracheal tube. A second method is to use blind intubation, manipulating the tube and/or the patient's head and neck. A third method employs flexible fiber-optic bronchoscope to both guide and visually confirm the proper placement of the endotracheal tube. Regardless of whether oral or nasal intubation is performed, the glottis must eventually be negotiated, and so illumination and visualization are highly desirable either way.

Several visualization and/or illumination devices have been produced and are in commercial use. For example, Aaron Medical Industries produces a lighted intubation guide that is essentially a thin stylet (wand) having a bright light at the tip. This stylet is placed within the endotracheal tube. As the endotracheal tube with its contained stylet is guided (blindly) through the pharynx, the operator of the tube may judge the approximate location of the tip of the endotracheal tube by observing the location of light transmitted through the patient's neck. This device provides no direct visualization of the glottis, and supplies only a small improvement over blind intubation. The Volpi Corporation manufactures a stylet device that provides direct visualization of the glottis. The device is placed within the endotracheal tube and uses fiber-optic bundles to transmits visual information from the tip of the tube back to the operator of the device. This device does not have its own light-source, but requires a separate light source. Another endotracheal intubation device and methods of using it is described in a paper by Hikaru Kohase “Endotracheal Intubation Device with a Charge Couple Device Camera” (Anesth. Analg. 2003: 96: 432-434). The device comprises a wand with a Charge Couple Device (CCD) camera mounted at the distal end, and includes a side tube through which a tube introducer is inserted. The introducer is positioned into the trachea through the vocal cords, and the wand is then withdrawn, leaving the introducer in place. Vitaid Airway Management Corporation sells the GlideScope™ device, which embeds a video camera and an Light Emitting Diode (LED) light source within a laryngoscopic blade. This device does not fit inside an endotracheal tube.

Olympus and Pentax corporations both produce flexible fiber-optic bronchoscopes that include both image-transmitting and light-transmitting fiber-optic bundles, as well as a fully articulated and guidable tip. These devices, while originally designed for bronchoscopy (the visualization of the lung bronchi) may also be used for difficult intubations as follows. First, the distal portion of the fiber-optic bronchoscope is inserted within an endotracheal tube. Then, by looking through an eyepiece on the bronchoscope while manipulating the endotracheal tube, the operator of the device is able to directly visualize the placement of the tube within the trachea. When the endotracheal tube is successfully placed, the fiber-optic device is withdrawn from the tube. Used in this way, the fiber-optic bronchoscope is referred to as a fiber-optic laryngoscope (not to be confused with the blade-like regular laryngoscope described earlier). Use of such fiber-optic devices provides a considerable improvement over blind intubation, but these devices are very complex and expensive, and require extensive training for effective use.

The patent literature includes a number of devices for insertion into the oral cavity that provide illumination and visualization. One of the earliest examples is U.S. Pat. No. 1,246,339 (Isaac Smit, 1917) that discloses a tongue depressor having an internal electric light source and a glass light-conducting element that allows light to be conducted from the bulb to the tip of the instrument to aid in visualization of the oral cavity.

A more recent disclosure is U.S. Pat. No. 6,655,377 (to Pacey) which describes an endotracheal intubation instrument having a camera and a light positioned near the tip of the instrument. The camera and light may be powered by a battery internal to the handle of the device. The camera is optionally a CCD or CMOS (Complementary Metal Oxide Semiconductor) camera and the light source is optionally an LED. Suction is provided near the tip of the device to cool the light source and to remove moisture that would otherwise cloud the camera lens. The visualization elements are not designed to fit within an endotracheal tube, but are mounted outside and adjacent to a tube.

U.S. Pat. No. 6,652,453 (to Smith) describes a self-contained, light-weight laryngoscope that includes a digital camera and “light emitters” both positioned close to the distal end of the scope, powered by an internal battery. The device includes a clamp at the end that grasps the endotracheal tube to be guided into place. As above, this device is not designed to fit within an endotracheal tube.

U.S. Pat. No. 6,322,498 (to Gravenstein) describes a tracheal imaging scope with a CCD camera and an LED light positioned at the proximal end of the instrument (near the operator) and uses fiber-optics to transmit light and images between the distal end of the instrument and the camera/light. Simple electrical and/or optical “quick-connectors” are used to link the components and the camera and light(s) are powered by an external power source. The device may include an lumen for ventilation, irrigation or suction, but is not designed to fit within an endotracheal tube.

U.S. Pat. No. 5,842,973 (to Bullard) describes a self-contained nasal-endotracheal intubation device with an “optical channel” connected to a camera and a “light channel” connected to an internal light source. Power is supplied by an internal battery. This device may be placed within an endotracheal tube and used to guide it into place.

U.S. Pat. No. 3,677,262 (to Zukowski) describes an illuminated endotracheal tube inserter with a light source and fiber-optic viewing bundle. This inserter device is designed to fit within an endotracheal tube.

U.S. Pat. No. 5,329,940 (to Adair) describes a hand-held endotracheal tube insertion device that includes fiber-optic cables for transmitting light and images. The device includes a malleable “insertion section” and in use, a standard endotracheal tube is fitted over the insertion section and removably attached to the handle of the device to allow visualization and insertion of the endotracheal tube into the trachea. An inflatable cuff, of a type that is standard on most endotracheal tubes, is provided near the distal end of the device which, when in use, is inflated to seal the endotracheal tube in the trachea and properly position the tip of the tube above and between the two bronchi.

U.S. Pat. No. 4,337,761 (to Upsher) describes a laryngoscope with a curved blade that removably grasps an endotracheal tube. The blade additionally possesses a light source and a fiber-optic viewing member to permit visualization of the epiglottis and larynx. Power is supplied by a battery in the handle. The blade can be flexible so that it may be bent into various curvatures suitable to the anatomy or a particular patient.

U.S. Pat. No. 5,676,598 (to Rudischhauser) describes a laryngoscope with a curved spatula blade where the blade includes a waveguide for transmitting light and a separate image waveguide for transmitting images.

U.S. Pat. No. 6,629,924 (to Adydelotte) describes an “enhanced endotracheal tube” with a fiber-optic light bundle and a reflectively coated bore used to transmit images to the user. Additionally an air passage is provided for inflating an inflatable cuff for positioning the device.

U.S. Pat. No. 6,146,402 (to Munoz) describes an endotracheal tube guide introducer that can be used to introduce a flexible guide tube into the trachea. Once in place, the guide tube is used to guide an endotracheal tube to its target. The device includes a fiber-optic visualization path as well as a light path for illuminating and viewing the epiglottis and larynx during use.

U.S. Pat. No. 5,665,052 (to Bullard) is another patent that describes an endotracheal tube guide. The guide is positioned in the trachea and an endotracheal tube is advanced along the guide to the desired location. Fiber-optic cables provide transmission of light and images.

U.S. Pat. No. 4,086,919 (to Bullard) discloses a laryngoscope for endotracheal intubation having a housing containing a working channel for containing forceps and channels containing fiber optics for lighting and viewing the internal areas of the body, and a laryngoscope blade for manipulating the epiglottis of a patient to enable viewing of a target area.

U.S. Pat. No. 3,766,909 (to Ozbey) describes a laryngoscope with a disposable blade and light guide. The light guide is incorporated into the blade and transmits light from a bulb in the handle. The bulb is powered by a battery, also located in the handle. The blade is designed to be cheap to manufacture and to be optionally disposable.

Visualization stylets, endotracheal guides and fiber-optic laryngoscopes and bronchoscopes were originally designed for bronchoscopy (visualization of the bronchi of the lungs), not for endotracheal intubation, and they generally suffer from a number of disadvantages. They are often complex and expensive to manufacture, requiring specialized parts fabrication and assembly. Due to their cost, they are generally non-disposable, which means that they have to be sterilized and carefully maintained after each use. This adds to the cost of maintaining such a device. They are generally difficult to sterilize due to the number and complexity of their sub-components and may require special procedures for cleaning and sterilization. They are frequently fragile, and fiber-optic light bundles are especially susceptible to damage. Repair is costly and takes the instrument out of use. One of the main problems in the use of the fiber-optic laryngoscope/bronchoscope is a reflection of one of its benefits, that is, its flexibility. Because of its flexibility and complicated controlling system, it is often difficult to control the bronchoscope as it is advanced through the patient airways to the vocal cords. The proper use of such fiber-optic devices requires significant training and it is estimated that 25 to 50 practice intubations on a mannequin followed by 50 to 100 intubations on normal patients is required before a physician should attempts what is termed “difficult airway management.” Because of these disadvantages, the financial cost of endotracheal intubation in patients who cannot be intubated solely through use of a regular laryngoscope (e.g., obese patients) is very high. In addition, significant delays in treatment may be caused by the need to locate and mobilize appropriate fiber-optic equipment. A need exists for a small, hand-held endotracheal visualization stylet that provides high quality optics and that is both easy to use and inexpensive to manufacture.

### GENERAL DESCRIPTION OF THE INVENTION

The present invention encompasses a visualization stylet. The visualization stylet of the invention may be used for various medical procedures including endotracheal intubation or to visualize the internal features of any anatomical structure such as the colon, vagina, esophagus, nasal passages, ear passages, or abdominal cavity.

In a preferred embodiment, the visualization stylet of the invention is used to facilitate endotracheal intubation. The visualization stylet is shaped and sized so that it may fit inside an endotracheal tube designed for endotracheal intubation of a human or animal subject. The stylet is elongated and preferably curved, and comprises a number of elements including a thin, flexible tube-shaped body defining a lumen therethrough, having a proximal end (near the operator) and a distal end (further away from the operator). The stylet also includes an image-gathering device, such as a charged couple device (CCD) or a complementary metal oxide semicondutor (CMOS) or a very large scale integrated (VLSI) chip camera, at or near the distal tip of the body, and a light-emitting device such as an LED or plurality of LEDs, also at or near the distal tip of the body. Electronic connectors transfer power and data to and from the image-gathering and light-emitting devices.

In use, the visualization stylet is placed within an endotracheal tube, such that the tip of the stylet is at the distal tip of the endotracheal tube, and the electronic connectors of the stylet are accessible from the proximal end of the endotracheal tube. The visualization stylet may optionally be reversibly attached in place relative to the endotracheal tube by using a standard luer-lock feature. The electronic connectors are attached to a power supply and a cathode ray tube (CRT) or equivalent device (e.g. an Liquid Crystal Display (LCD) monitor), thus providing a view of the patient's pharynx, glottis, and other anatomical structures during intubation. Once intubation is accomplished, the visualization stylet is withdrawn from the endotracheal tube and either sterilized for re-use, or preferably discarded.

The light source may be of any acceptable type; for example, it may be an incandescent electric light or preferably a light emitting diode (LED). The light source is generally mounted at the distal end of the stylet and is preferably positioned and shielded in such a way that the illumination from the light source does not interfere with the image received by the camera. In one embodiment, the light source is positioned in front of the camera and is shielded from the camera (for example, by the rim of a collimator) so that the light projects forward from the device and not backward towards the camera. Light may optionally be supplied by a light source separate from the device, wherein the light is transmitted to the distal tip of the stylet by means of fiber-optic cables. The stylet may employ a single light source such as an LED or a plurality of LEDs. Such LEDs may optionally be arranged in a generally circular pattern about the distal tip of the stylet.

The camera may be any suitable image collecting device known in the art, for example a charged couple device (CCD) or other electronic camera may be used. The image received by the camera may be transmitted directly from the illuminated object or may be transmitted and focused from the illuminated object to the camera via a lens (or plurality of lenses).

Additionally, an optional collimator may be positioned in front of the lens. One or more LEDs may be mounted peripherally to the collimator, so that the collimator shields the camera from the light emitted by the LEDs. The collimator both improves the optics of the system by filtering non-parallel incoming light, and shields the camera from direct illumination by the light source(s).

In an alternative embodiment the stylet is provided with more than one camera. In particular, the provision of two adjacent cameras enables stereoscopic imaging. In a stereoscopic embodiment where lenses are used, the device may include a lens for each camera. Each camera may have one or more associated lenses. Each camera may optionally have its own lens(es) and its own collimator.

In this disclosure the term “lens” includes any transparent cover, whether or not it can serve to focus light, and specifically includes transparent covers whose sole purpose is to protect the image-gathering device (e.g., a camera).

In order to keep the front lens of the camera free of condensation, fluids, mucus or other debris, the device may also include one or more of the following. It may include a moisture-removing element such as a heating element in thermal communication with the lens to keep the lens free of moisture or a vacuum or suction device. It may include a debris-removing element to remove solid or liquid debris, such as a vacuum or suction device, or a lens-washing element or an air-jet or water-jet device, or a mechanical wiper device. These components may be activated at the will of the operator to maintain a clear view. Such devices are well-known and may be adapted for use with the invention.

Alternatively, fiber optic cables maybe used to transmit visual information from the illuminated object to the camera which may be placed at or near the proximal end of stylet, or may be located separately from the stylet. In a preferred embodiment, the camera is mounted at or near the distal end of the stylet in such a way that it may receive visual information from the illuminated object without interference from the light source.

In certain embodiments one or more working channels may also be included in the stylet. Such a working channel can receive a flexible guide member which in use may be passed through the working channel and guided through the vocal chords into the trachea prior to introduction of an intubation apparatus into the subject. Alternatively, the working channel may be used to receive a catheter or may be used for suction, delivery of oxygen or other gases, or delivery of local and/or general anesthetics to the subject.

In another embodiment, the distal tip of the stylet may be controllable by the operator and may be pivoted in two or three dimensions to allow additional visualization of internal structures. Methods of achieving such manipulation are known and described for instance in U.S. Pat. No. 5,318,008 and 5,842,973.

The stylet tube of the invention may be made from any suitable material that is malleable such that it may be bent into a shape suitable for introduction into the anatomy of a particular space such as the oral cavity and larynx. Suitable materials for making the stylet tube are well known in the catheter art and include metals such as aluminum, plastics and polymers such as polyvinylchloride, polypropylene, polyethylene, polyester, polyamide and silicone. Such materials are simple to manufacture in various shapes and sizes and are easy to sterilize.

The stylet may include an internal power supply, such as a battery. In certain embodiments, such as when the light source and/or camera can function using very low electrical current, standard disposable dry cell batteries may be used to power both camera and lights. Batteries may be contained within the structure of the stylet, or located externally and connected via standard electrical connections.

In embodiments where power is supplied externally to the stylet, a standard electrical coupling may be used to transmit power from an external electrical source such as a battery or transformer. Visual signals are transmitted from the device's camera to a display screen, such as a liquid crystal display (LCD) or cathode ray tube (CRT), and such signals may be transmitted via a standard optical or electrical cables. Visual information may be stored in an analog or digital storage device for later retrieval.

The visualization stylet of the invention displays several advantageous characteristics including the fact that it is inexpensive to manufacture because it may be constructed from standard electrical components such as LEDs, CCD cameras, and other standard electrical components. The cost of construction may be sufficiently small such that the device may effectively be disposable. If disposable, then the device requires no sterilization, reducing the cost of operation. The visualization stylet is also rugged and, because of its relative simplicity, is less prone to malfunction and damage than presently-used devices. Ease and effectiveness of use reduces the incidence of trauma to the patient and increases intubation speed, which may be life-saving. Additionally the stylet of the invention provides high quality optics and is easy to use without specialized training.

Further embodiments, objects and advantages of the invention will become apparent from the following detailed descriptions, examples, drawings and claims.

### BRIEF DESCRIPTION OF THE FIGURES

FIG. 1 is a schematic longitudinal cross-sectional representation of a general embodiment of the visualization stylet.

FIG. 2 is a schematic representation of the visualization stylet fitted within an

FIG. 3 is a schematic representation of a stereoscopic visualization stylet employing two separate cameras and two lenses.

FIG. 4 is a schematic representation of the visualization stylet of the invention fitted with an optional collimator.

### KEY TO COMPONENT NUMBERS

1. Stylet Tube

2. Lens

3. Light source

4. Camera

5. Power supply conduit for light source

6. Power supply conduit for light camera

7. Data transmission conduit for camera

8. Power supply for light

9. Video display

10. Power supply for camera

11. Endotracheal tube

12. Endotracheal cuff

13. Inflation cuff

14. Visualization stylet

15. Second camera

16. Second lens

17. Collimator

### DETAILED DESCRIPTION OF EXEMPLARY EMBODIMENTS

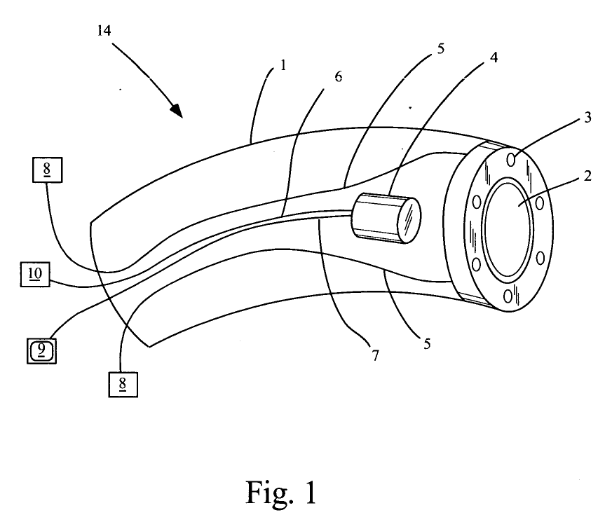

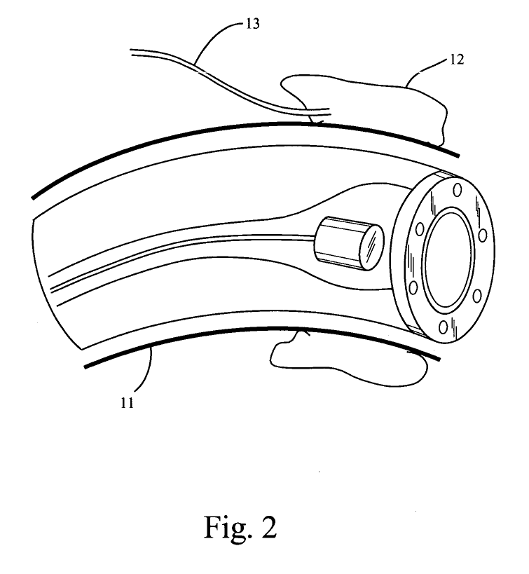

A specific embodiment of the invention is shown in FIGS. 1 and 2. FIG. 1 shows a schematic representation of the visualization stylet (14). All the elements of the stylet are contained within the lumen of the stylet the (1). The stylet in this particular embodiment has a plurality of white LED lights (3) disposed in a circular pattern at the outside circumference of the distal tip of the stylet, surrounding a central lens (2). The lens focuses light from an image onto the CCD camera (4). The LED lights receive power from one or more power conduits (5) which are electrically connected to a power supply (8). The power supply may be one or more dry cell batteries contained within the body of the stylet, or may be external. The camera, which may be a CCD camera, is centered within the axis of the lumen and slightly behind the distal tip of the stylet tube (1), shielded from the lights (3). The camera receives electrical power from a power supply (10) via a power supply conduit (6) and transmits visual information to a video display (9) via the data transmission conduit (7). The power supply to the camera and to the LED lights may be identical, depending on the voltage/power requirements of the camera and the LED lights.

In this embodiment, the body of the visualization stylet (14) is formed from a hollow malleable stylet tube (1). The stylet tube may be made of any suitable material that is plastic in nature, i.e., that maintains the shape into which it is bent. In a preferred embodiment the body is made out of a synthetic shape-retaining material. In general, aluminum, brass, plastic, or any other shape-retaining materials such as polyvinylchloride, polypropylene, polyethylene, polyester, polyamide, and silicone may be used.

The stylet may be straight or the distal portion of the stylet may curved. In a curved embodiment, the distal portion (approximately the distal 2 to 10 inches) may be evenly curved through an angle of between 2 degrees and 45 degrees, preferably between 5 degrees and 22 degrees, or between 7 degrees and 15 degrees. The portion of the stylet that is curved may be different for different anatomies, for example, for a baby, the stylet mat be curved only at the terminal 1 to 3 inch portion. The diameter of the stylet is appropriate so that it fits within the lumen of the endotracheal tube, thus the diameter may be from about 0.5 centimeters to about 2.5 centimeters, or about 1.0 centimeter to about 2.0 centimeters or preferably is from about 8 millimeters to about 12 millimeters.

At the distal tip of the visualization stylet (14) one or a plurality of light sources (3) is provided. In a preferred embodiment, the light sources are disposed in a circular pattern at the outside circumference of the distal tip of the stylet. The light sources are preferably white LED lights, but may be incandescent or fluorescent lights or in another embodiment may be a non-coherent light source transmitted via a fiber-optic bundle. The light sources project light forward from the tip of the stylet during intubation which illuminates the objects to be viewed. Incoming light rays reflected from the object to be viewed are focused through the lens or lenses (2) onto the camera (4).

The camera is preferably a Charge Coupled Device (CCD) of a type commonly used in digital cameras. The camera receives power via a power supply conduit (5) and transmits an electrical signal via a data transmission conduit (7) to a video display screen (9) such as an LCD or CRT screen. The operator views the screen to monitor the progress of the endotracheal tube through the vocal chords into the trachea.

The power and data-transmission conduits run within the lumen of the stylet and project out from the proximal end of the stylet, terminating in standard video output and power input couplings which are operatively attached to the video screen and the power source, respectively. If the device contains an internal battery, then only a video output need project from the proximal end of the device. Depending on the voltage requirements of the camera and LEDs, a single power supply (either internal or external battery) may be used to power both the camera and the LEDs. In an embodiment where light is transmitted via fiber optic cables, a non-coherent fiber-optic bundle runs through the stylet tube from the light source to the distal tip of the stylet.

FIG. 2 is a schematic diagram showing the visualization stylet (14) fitted within the lumen of an endotracheal tube (11), showing the endotracheal cuff (12) deflated. The cuff is a flexible balloon toroidaly attached about the outer surface of the distal end of the endotracheal tube (11) and is in air/fluid communication with an inflation tube (13). In use, the cuff is inflated by providing a positive pressure via the inflation tube; the cuff serves both to hold the endotracheal tube in place and to prevent passage of stomach or oropharngeal contents into the lungs.

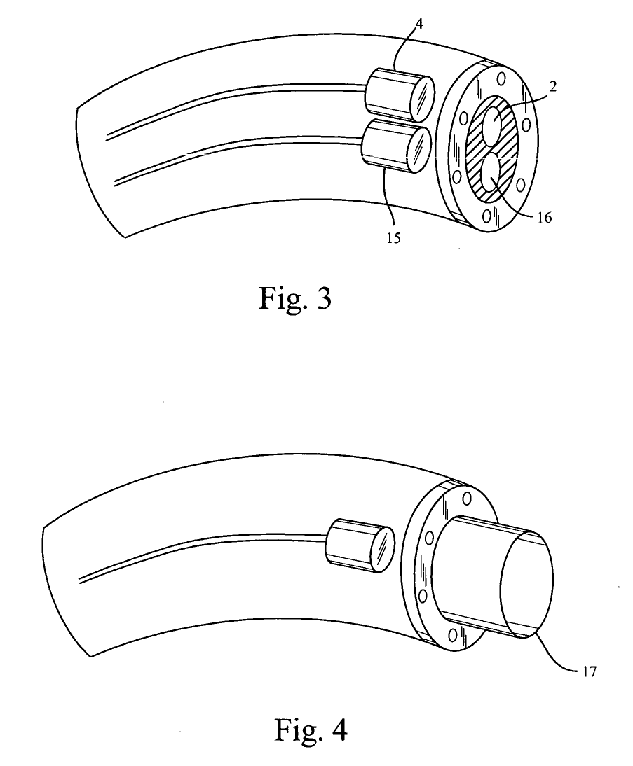

FIG. 3 shows a variation of the invention in which a stereoscopic image is provided by means of two cameras mounted side by side. In certain stereoscopic embodiments, lenses may be used to focus the light from objects into the cameras. The number of lenses will generally equal the number of cameras. In the example shown in FIG. 3, there are two cameras and two lenses. Alternatively, in place of the lenses, a transparent window may be provided to prevent fluid and other matter from fouling the camera(s). Such a window may be made of glass or Perspex or any other suitable material. In yet other embodiments, no lens or window is provided. Images are transmitted via the cameras and may be displayed on a screen using differential color imaging. Such images may be viewed by the operator using 3-D goggles to give the effect of a three-dimensional image. Alternatively, the separate images may be processed by a computer to produce a three dimensional image that may be displayed and perceived without the need for special 3-D glasses. In another embodiment, a stereoscopic image may be provided without the need for a second camera. This may be done by splitting the single image into two images using an optical path separator and conducting each image to a separate camera. Such an embodiment may employ, for example, a single glass or plastic optical rod element to capture the initial single image, a prismatic optical path separator mounted behind the rod lens, and dual charged-coupled devices to capture stereoscopic images. Video images can be processed electronically to convey images to a head-mounted display. See Neurosurgical Focus 6 (4):Article 12, 1999; and Eguchi S et al. “Stereoscopic Ophthalmic Microendoscope System” Arch. Ophthalmol. 115:1336-1338, 1997.

FIG. 4 shows an alternate embodiment employing a collimator to shield the light from the light sources from the camera. The collimator in the figure is somewhat exaggerated and need only be of a size and shape sufficient to shield the camera. In the example shown, the collimator is a hollow tube that projects from the distal tip of the stylet. The light sources (LEDs) are mounted circumferentially about the collimator, while the camera is positioned slightly back from the tip of the stylet and within the central lumen of the stylet tube. In other embodiments, separate collimators may be positioned over and around the individual light sources, forming a tube around the light that restricts the peripheral dispersion of the light so that only the desired target is illuminated.

In use, the visualization stylet (14) is inserted into a standard endotracheal tube (11) such that the tip of the stylet is at or near the distal tip of the endotracheal tube. The power supply conduit (if both the camera and LEDs are powered by the same supply, which is preferable) or conduits (if the camera and LEDs require a separate supply)(5 and 6) and the data transmission conduit (7) project from the proximal end of the endotracheal tube. The power supply conduit or conduits are operatively attached to appropriate power supplies (either internal battery, or external) and the data transmission conduit is communicably attached to a screen (e.g., LCD or CRT), thus providing a view of the patient's pharynx, glottis, and other anatomical structures during intubation. Once intubation is accomplished, the visualization stylet is withdrawn from the endotracheal tube and either sterilized for re-use, or preferably discarded.

The embodiments disclosed in this document are illustrative and exemplary and are not meant to limit the invention. Other embodiments and equivalents can be utilized and structural changes can be made without departing from the scope of the claims of the present invention. The present invention encompasses all embodiments and variations claimed and all equivalents thereof. As used herein and in the appended claims, the singular forms “a,” “an,” and “the” include plural reference unless the context clearly dictates otherwise.

1. A visualization stylet comprising: a substantially cylindrical elongated stylet tube defining a longitudinal lumen therethrough, the stylet tube having a proximal end and a distal end;
an image gathering device at least partially positioned within the lumen of the stylet tube at or near the distal end of the stylet tube wherein the image gathering device is communicably attached to a visual display device; and
a light source disposed at or near the distal end of the stytlet tube wherein the light source is electrically attached to a power source.

2. The visualization stylet of claim 1 wherein the stylet is shaped and sized to fit within the lumen of an endotracheal tube for the endotracheal intubation of a human subject.

3 The visualization stylet of claim 1 wherein the stylet is shaped and sized to fit within the lumen of an endotracheal tube for the endotracheal intubation of a non-human subject.

4. The visualization stylet of claim 2 wherein the distal portion of the stylet is curved.

5. The visualization stylet of claim 2 wherein the distal portion of the stylet is curved through an angle of between 5 degrees and 22 degrees.

6. The visualization stylet of claim 2 further comprising a plurality of light sources disposed at or near the distal end of the stylet tube.

7. The visualization stylet of claim 2 wherein the light source comprises a light-emitting-diode.

8. The visualization stylet of claim 2 wherein the light source comprises a fiber optic conduit.

9. The visualization stylet of claim 2 wherein the image gathering device comprises a device selected from the group consisting of: a charged couple device (CCD), complementary metal oxide semiconductor (CMOS), and a very large scale integrated (VLSI) chip.

10. The visualization stylet of claim 2 wherein the image gathering device comprises a fiber optic conduit.

11. The visualization stylet of claim 2 further comprising at least one lens positioned at or near the distal end of the stylet tube and distal to the image gathering device.

12. The visualization stylet of claim 11 further comprising a moisture-removing element positioned near the distal tip of the stylet tube, in close proximity to said at least one lens, the moisture-removing element selected from the group consisting of: a heating element, a vacuum device, and a suction device.

13. The visualization stylet of claim 11 further comprising a debris-removing element positioned near the distal tip of the stylet tube, in close proximity to said at least one lens, the debris-removing element selected from the group consisting of: a vacuum device, a suction device, a lens-washing device, an air-jet device, a water-jet device, and a mechanical wiper device.

14. The visualization stylet of claim 2 further comprising a power source disposed within the lumen of the stylet tube.

15. The visualization stylet of claim 2 further comprising at least one working channel.

16. The visualization stylet of claim 15 wherein the working channel is adapted to receive a guide member.

17. The visualization stylet of claim 15 wherein the working channel is adapted to conduct a fluid.

18. The visualization stylet of claim 15 wherein the working channel is adapted to conduct an anesthetic.

19. The visualization stylet of claim 2 wherein the distal tip of the stylet is maneuverable.

20. The visualization stylet of claim 2 further comprising a second image gathering device whereby stereoscopic imaging is provided.

21. The visualization stylet of claim 20 further comprising at least one lens positioned at or near the distal end of the stylet tube and distal to at least one image gathering device.

22. The visualization stylet of claim 2 further comprising a collimator projecting from the distal end of the stylet in such a disposition as to shield the image gathering device from the light source.

23. The visualization stylet of claim 2 wherein the stylet is shaped and sized to fit within the lumen of a tube adapted for nasopharyngeal intubation.
